(19) United States
(12) Patent Application Publication
(10) Pub. No.: US 2004/0048822 A1
(43) Pub. Date: Mar. 11, 2004

(54) Nucleic acid immunization

(76) Inventor: Juan Harrison, Fremont, CA (US)

Correspondence Address: BURNS DOANE SWECKER & MATHIS L L P, POST OFFICE BOX 1404, ALEXANDRIA, VA, 22313-1404, US

(21) Appl. No.: 10/415,745

(22) PCT Filed: Nov. 1, 2001
(86) PCT No.: PCT/US01/50673

(51) Int. Cl. A61K 48/00; A61K 9/16; A61K 9/50

(52) U.S. Cl. 514/44; 424/490

(57) ABSTRACT

Polynucleotides encoding T-cell receptors and compositions and methods of use these polynucleotides are provided. The invention includes polynucleotides encoding at least one T-cell receptor or fragments thereof, core carriers coated with these polynucleotides and methods of treating T-cell mediated diseases in a subject.

### FIELD OF THE INVENTION

The present invention relates generally to polynucleotides encoding T-cell receptors, compositions containing such polynucleotides, and methods of use thereof. More particularly, the invention pertains to polynucleotides encoding at least one T-cell receptor molecule or a fragment thereof, particulate pharmaceutical compositions containing such polynucleotides, and methods of treating T-cell mediated diseases in a subject by administering the particulate pharmaceutical compositions of the present invention to elicit a cross-reactive immune response against a specific population of T cells that express the T-cell receptor.

### BACKGROUND

Recently, direct injection of plasmid DNA by intramuscular (Wolff et al. (1990) Science 247:1465:1468) or intradermal injection with a needle and syringe (Raz et al. (1994) PNAS USA 91:9519-9523) has been described. Another approach referred to as Biolistic®, or particle-mediated DNA delivery employs a needless-device to deliver DNA-coated microscopic gold beads directly into the cells of the epidermis. (Yang et al. (1990) PNAS USA 87:9568-9572). Thus, a number of delivery techniques can be used to deliver nucleic acids for immunizations, including particle-mediated techniques which deliver nucleic acid-coated microparticles into target tissue. (see, e.g., co-owned U.S. Pat. No. 5,865,796, issued Feb. 2, 1999). Particle-mediated nucleic acid immunization techniques have been shown to elicit both humoral and cytotoxic T lymphocyte immune responses following epidermal delivery of nanogram quantities of DNA. Pertmer et al. (1995) Vaccine 13:1427-1430. Such particle-mediated delivery techniques have been compared to other types of nucleic acid inoculation, and found markedly superior. Fynan et al. (1995) Int. J. Immunopharmacology 17:79-83, Fynan et al. (1993) Proc. Natl. Acad. Sci. USA 90:11478-11482, and Raz et al. (1994) Proc. Natl. Acad. Sci. USA 91:95199523.

A novel transdermal injection system that entails the use of a needleless syringe to deliver solid drug-containing particles in controlled doses into and through intact skin has also been described. In particular, commonly owned U.S. Pat. No. 5,630,796 to Bellhouse et al., describes a needleless syringe that delivers pharmaceutical particles entrained in a supersonic gas flow. The needleless syringe (also referred to as “the PowderJect® needleless syringe device”) is used for transdermal delivery of powdered drug compounds and compositions, for delivery of genetic material into living cells (e.g., gene therapy) and for the delivery of biopharmaceuticals to skin, muscle, blood or lymph. The needleless syringe can also be used in conjunction with surgery to deliver drugs and biologics to organ surfaces, solid tumors and/or to surgical cavities (e.g., tumor beds or cavities after tumor resection). Pharmaceutical agents that can be suitably prepared in a substantially solid, particulate form can be safely and easily delivered using such a device.

One particular needleless syringe generally comprises an elongate tubular nozzle having a rupturable membrane initially closing the passage through the nozzle and arranged substantially adjacent to the upstream end of the nozzle. Particles of a therapeutic agent to be delivered are disposed adjacent to the rupturable membrane and are delivered using an energizing means which applies a gaseous pressure to the upstream side of the membrane sufficient to burst the membrane and produce a supersonic gas flow (containing the pharmaceutical particles) through the nozzle for delivery from the downstream end thereof. The particles can thus be delivered from the needleless syringe at delivery velocities of between Mach 1 and Mach 8 which are readily obtainable upon the bursting of the rupturable membrane.

Another needleless syringe configuration generally includes the same elements as described above, except that instead of having the pharmaceutical particles entrained within a supersonic gas flow, the downstream end of the nozzle is provided with a bistable diaphragm which is moveable between a resting “inverted” position (in which the diaphragm presents a concavity on the downstream face to contain the pharmaceutical particles) and an active “everted” position (in which the diaphragm is outwardly convex on the downstream face as a result of a supersonic shockwave having been applied to the upstream face of the diaphragm). In this manner, the pharmaceutical particles contained within the concavity of the diaphragm are expelled at a high initial velocity from the device for transdermal delivery thereof to a targeted skin or mucosal surface.

Transdermal injection using the above-described needleless syringe configurations is carried out with particles having an approximate size that generally ranges between 0.1 and 250 μm. Particles larger than about 250 μm can also be delivered from the device, with the upper limitation being the point at which the size of the particles would cause untoward damage to the skin cells. The actual distance which the delivered particles will penetrate depends upon particle size (e.g., the nominal particle diameter assuming a roughly spherical particle geometry), particle density, the initial velocity at which the particle impacts the skin surface, and the density and kinematic viscosity of the skin. Target particle densities for use in needleless injection generally range between about 0.1 and 25 g/cm³, and injection velocities generally range between about 150 and 3,000 m/sec.

### SUMMARY OF THE INVENTION

The present invention provides methods and compositions relating to immunization of a vertebrate subject in order to treat a specific T cell mediated disease or disorder, wherein the compositions contain a polynucleotide encoding a T-cell receptor (TCR) or a relevant fragment thereof, and the TCR is found on (expressed by) specific T cells that are preferentially associated with T cell mediated disorder such as an autoimmune disorder.

It is thus a primary object of the invention to provide an isolated polynucleotide, wherein the polynucleotide comprises a coding sequence for at least one T-cell receptor (TCR) operably linked to control elements such that the coding sequence can be transcribed and translated in vivo in a recipient cell to provide a properly folded (e.g., a refolded) TCR molecule. The isolated polynucleotide is used in the manufacture of a medicament for eliciting a cross-reactive immune response in a vertebrate subject against a T cell expressing a native form of the TCR. The medicament is a particulate medicament that is suitable for administration to the subject using a transdermal particle injection technique. Accordingly, in one particular embodiment, the medicament is formed by coating a core carrier with the polynucleotide. In another embodiment, the medicament is a solid, particulate pharmaceutical composition containing the polynucleotide. In certain embodiments, the polynucleotide encodes a TCR molecule comprising an a chain and a chain. In other embodiments, the polynucleotide encodes a TCR molecule comprising a y chain and a δ chain. The TCR-encoding polynucleotides of the invention are selected on the basis that T cells expressing the TCR are preferentially associated with an autoimmune disorder, including, for example, multiple sclerosis, psoriasis, rheumatoid arthritis and lupus.

It is also a primary object of the invention to provide an isolated polynucleotide, wherein the polynucleotide comprises a coding sequence for a TCR fragment, for example the coding sequence for a CDR2 hypervariable region of a TCR, wherein the CDR2 is from a T cell preferentially associated with an autoimmune disorder. The isolated polynucleotide is used in the manufacture of a medicament for eliciting a cross-reactive immune response in a vertebrate subject against a T cell expressing a native form of the TCR. The medicament is a particulate medicament that is suitable for administration to the subject using a transdermal particle injection technique. Accordingly, in one particular embodiment, the medicament is formed by coating a core carrier with the polynucleotide. In another embodiment, the medicament is a solid, particulate pharmaceutical composition containing the polynucleotide. In certain embodiments, a second polynucleotide is also provided in the medicament, wherein the second medicament comprises a coding sequence for at least one further TCR hypervariable region, for example a coding sequence for a CDR1 and/or a CDR3 peptide. Preferably, the T cells expressing the TCRs or fragments thereof are preferentially associated with an autoimmune disorder (e.g., multiple sclerosis, psoriasis, rheumatoid arthritis and lupus).

It is a still further primary object of the invention to provide a method for treating a T-cell mediated disease or disorder. The method entails administration of one of the particulate medicaments of the present invention to a vertebrate subject, whereby the polynucleotide is expressed in recipient cells of the subject to provide a TCR molecule or a fragment thereof (e.g., a CDR2 peptide) in an amount sufficient to induce a cross-reactive immune response against specific T cells expressing the homologous TCR, wherein those T cells mediate the pathology associated with the T cell disorder or disease. Preferably, the TCRs or fragments thereof are from T cells that are preferentially associated with an autoimmune disorder (e.g., multiple sclerosis, psoriasis, rheumatoid arthritis and lupus). In certain embodiments, the polynucleotide encodes a TCR molecule comprising an a chain and a β chain. In other embodiments, the polynucleotide encodes a TCR molecule comprising a γ chain and a δ chain. In addition, the medicament can include one or more additional polynucleotides, wherein the additional polynucleotides can encode full-length TCR molecules or fragments thereof (e.g., CDR1, CDR2 and/or CDR3 peptides). In specific embodiments, the additional polynucleotides are coated onto a core carrier (e.g., gold or tungsten) and the carrier is administered to a skin cell or to a mucosal cell in the subject being treated.

It is yet another primary object of the invention to provide methods that further comprise the step of identifying nucleotide sequences of T-cell receptor types that are preferentially expressed at the site of disease and selecting one or more of the identified nucleotide sequences for inclusion in the polynucleotides described herein. In certain embodiments, the identification comprises identifying nucleotide sequences of CDR2 regions that are preferentially expressed in TCRs at the site of disease and selecting one or more of the identified nucleotide sequences for inclusion in the polynucleotide.

Accordingly, in one aspect of the invention a core carrier particle is provided. The core carrier is coated with a polynucleotide comprising a coding sequence for at least one T-cell receptor (TCR) operably linked to control elements such that the coding sequence can be transcribed and translated in vivo in a recipient cell to provide a refolded TCR molecule.

In a related aspect of the invention, a core carrier is provided, wherein the core carrier is coated with a polynucleotide comprising a coding sequence for a CDR2 hypervariable region of a T-cell receptor (TCR) operably linked to control elements such that the coding sequence can be transcribed and translated in vivo in a recipient cell to provide a CDR2 peptide, wherein the CDR2 is from a T cell preferentially associated with an autoimmune disorder.

In another aspect of the invention, a solid, particulate pharmaceutical composition is provided, wherein the composition contains a polynucleotide comprising a coding sequence for at least one T-cell receptor (TCR) operably linked to control elements such that the coding sequence can be transcribed and translated in vivo in a recipient cell to provide a refolded TCR molecule.

In a related aspect of the invention, a solid, particulate pharmaceutical composition is provided, wherein the composition contains a polynucleotide comprising a coding sequence for a CDR2 hypervariable region of a T-cell receptor (TCR) operably linked to control elements such that the coding sequence can be transcribed and translated in vivo in a recipient cell to provide a CDR2 peptide, wherein the CDR2 is from a T cell preferentially associated with an autoimmune disorder.

In still another aspect of the invention, a vector comprising a polynucleotide with a coding sequence for at least one T-cell receptor (TCR) is used in the manufacture of a medicament for eliciting a cross-reactive immune response against a T cell expressing a native form of the TCR, wherein the medicament is a particulate medicament suitable for transdermal injection into a subject. The coding sequence is operably linked to control elements such that it can be transcribed and translated in vivo in recipient cells of the subject to provide a refolded TCR molecule.

In a related aspect of the invention, a vector comprising a polynucleotide with a coding sequence for a CDR2 hypervariable region of a T-cell receptor (TCR) is used in the manufacture of a medicament for eliciting a cross-reactive immune response against a T cell expressing a native form of the TCR, wherein the CDR2 is from a T cell preferentially associated with an autoimmune disorder and the medicament is a particulate medicament suitable for transdermal injection into the subject. The coding sequence is operably linked to control elements such that it can be transcribed and translated in vivo in recipient cells of the subject to provide a CDR2 peptide.

In yet another aspect of the invention, a method is provided for treating a T-cell mediated disease. The method entails administering to a vertebrate subject in need of such therapy an effective amount of either: (a) a core carrier coated with a polynucleotide comprising a coding sequence for at least one T-cell receptor (TCR) operably linked to control elements such that the coding sequence can be transcribed and translated in vivo in a recipient cell to provide a refolded TCR molecule; or (b) a core carrier coated with a polynucleotide comprising a coding sequence for a CDR2 hypervariable region of a T-cell receptor (TCR) operably linked to control elements such that the coding sequence can be transcribed and translated in vivo in a recipient cell to provide a CDR2 peptide, wherein the CDR2 is from a T cell preferentially associated with an autoimmune disorder. After administration, the polynucleotide is expressed in cells of the subject to provide a T-cell receptor (TCR) molecule or a CDR2 peptide in an amount sufficient to induce a cross-reactive immune response against a native TCR expressed by T cells mediating the T-cell mediated disease.

In a related aspect of the invention, another method is provided for treating a T-cell mediated disease. The method entails administering to a vertebrate subject in need of such therapy an effective amount of either: (a) a solid, particulate pharmaceutical composition comprising a polynucleotide with a coding sequence for at least one T-cell receptor (TCR) operably linked to control elements such that the coding sequence can be transcribed and translated in vivo in a recipient cell to provide a refolded TCR molecule; or (b) a solid, particulate pharmaceutical composition comprising a polynucleotide with a coding sequence for a CDR2 hypervariable region of a T-cell receptor (TCR) operably linked to control elements such that the coding sequence can be transcribed and translated in vivo in a recipient cell to provide a CDR2 peptide, wherein the CDR2 is from a T cell preferentially associated with an autoimmune disorder. After administration, the polynucleotide is expressed in cells of the subject to provide a T-cell receptor (TCR) molecule or a CDR2 peptide in an amount sufficient to induce a cross-reactive immune response against a native TCR expressed by T cells mediating the T-cell mediated disease.

These and other objects, aspects and embodiments of the present invention will readily occur to those of ordinary skill in the art in view of the disclosure provided herein.

### MODES FOR CARRYING OUT THE INVENTION

Before describing the present invention in detail, it is to be understood that this invention is not limited to particular T-cell receptor coding sequences. It is also to be understood that different applications of the disclosed methods may be tailored to the specific needs in the art. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

It must be noted that, as used in this specification and the appended claims, the singular forms “a”, “an”, and “the” include plural referents unless the content clearly dictates otherwise. Thus, for example, reference to “a core carrier” includes a mixture of two or more such particles, reference to “a particle” includes reference to mixtures of two or more particles, reference to “a recipient cell” includes two or more such cells, and the like.

Definitions

Unless defined otherwise, all technical and scientific terms used herein have the same meaning as commonly understood by one of ordinary skill in the art to which the invention pertains. The following terms are intended to be defined as indicated below.

The term “transdermal” delivery intends intradermal (e.g., into the dermis or epidermis), transdermal (e.g., “percutaneous”) and transmucosal administration, i.e., delivery by passage of an agent into or through skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Thus, the term encompasses delivery from a particle delivery device as described in U.S. Pat. No. 5,630,796, as well as particle-mediated delivery as described in U.S. Pat. No. 5,865,796.

By “core carrier” is meant a carrier particle on which a nucleic acid (e.g., DNA) is coated in order to impart a defined particle size as well as a sufficiently high density to achieve the momentum required for cell membrane penetration, such that the DNA can be delivered using particle-mediated delivery techniques, for example those described in U.S. Pat. No. 5,100,792. Core carriers typically include materials such as tungsten, gold, platinum, ferrite, polystyrene and latex. See e.g., Particle Bombardment Technology for Gene Transfer, (1994) Yang, N. ed., Oxford University Press, New York, N.Y. pages 10-11.

By “needleless syringe” is meant an instrument which delivers a particulate composition transdermally, without a conventional needle that pierces the skin. Needleless syringes for use with the present invention are discussed throughout this document. The term thus includes particle delivery devices.

By “antigen” is meant a molecule which contains one or more epitopes that will stimulate a host's immune system to make a cellular antigen-specific immune response, or a humoral antibody response. Thus, antigens include proteins, polypeptides; antigenic protein fragments, oligosaccharides, polysaccharides, and the like. Furthermore, the antigen can be derived from any known virus, bacterium, parasite, plants, protozoans, or fungus, and can be a whole organism. The term also includes tumor antigens Similarly, an oligonucleotide or polynucleotide which expresses an antigen, such as in DNA immunization applications, is also included in the definition of antigen. Synthetic antigens are also included, for example, polyepitopes, flanking epitopes, and other recombinant or synthetically derived antigens (Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998).

The term “epitope” generally refers to the site on an antigen to which a specific antibody molecule binds. The identification of epitopes which are able to elicit an antibody response is readily accomplished using techniques well known in the art. See, e.g., Geysen et al. Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). T-cell epitopes are generally those features of a peptide structure capable of inducing a T-cell response. In this regard, it is accepted in the art that T-cell epitopes comprise linear peptide determinants that assume extended conformations within the peptide-binding cleft of MHC molecules, (Unanue et al., Science (1987) 236:551-557). As used here, an epitope is generally a peptide having about 3-5, preferably 5-10 or more amino acid residues.

The term “self antigen,” which is used interchangeably herein with the term “autoantigen,” means an antigen, or a molecule capable of being recognized during an immune response, that is normally part of the individual. This is in contrast with antigens which are foreign, or exogenous, which are not normally part of the individual's milieu. Each autoimmune disease is characterized by an immune response directed at a self antigen. Normally, there are no active immune responses to self antigens, and no symptoms appear. With the development of an immune response to a self antigen, autoimmune diseases may appear. Autoimmune diseases present clinically with different symptoms depending upon the specific self antigen against which an immune response is raised. This immune response results in the destruction of the structure containing the self antigen, and it is the loss of that structure with concurrent loss of that structure's normal function which results in symptoms of autoimmune disease.

A “peptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein.

The terms “nucleic acid molecule” and “polynucleotide” are used interchangeably to and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the linear representation of a polynucleotide molecule. This linear representation can be input into databases in a computer having a central processing unit and used for bioinformatics applications such as functional genomics and homology searching.

A “vector” is any moiety capable of transferring gene sequences to target cells (e.g., viral vectors, non-viral vectors, particulate carriers, and liposomes). A “plasmid” vector is an extrachromosomal genetic element which is capable of self-replication in a host cell. Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Transcription and translation of coding sequences are typically regulated by “control elements”, including, but not limited to, transcription promoters, transcription enhancer elements, Shine and Delagarno sequences, transcription termination signals, polyadenylation sequences (located 3′ to the translation stop codon), sequences for optimization of initiation of translation (located 5′ to the coding sequence), and translation termination sequences.

A “promoter” is a nucleotide sequence which directs transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), constitutive promoters, and selective promoters (where expression of a polynucleotide sequence operably linked to the promoter only occurs in particular tissue types). It is intended that the term “promoter” or “control element” includes full-length promoter regions and functional (e.g., controls transcription or translation) segments of these regions.

An “isolated polynucleotide” molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith. A sequence is “derived from” a molecule if it has the same or substantially the same basepair sequence as a region of the source molecule, its cDNA; complements thereof, or if it displays sequence identity as described below.

“Operably linked” refers to an arrangement of elements wherein the components so described are configured so as to perform their usual function. Thus, a given promoter that is operably linked to a coding sequence (e.g., encoding an antigen of interest) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 90%, and most preferably at least about 95%-98% sequence identity over a defined length of the molecules, as determined using the methods above. As used herein substantially homologous also refers to sequences showing complete identity to the specified DNA or polypeptide sequence. DNA sequences that are substantially homologous can be identified in a Southern hybridization experiment under, for example, stringent conditions, as defined for that particular system. For example, stringent hybridization conditions can include 50% formamide, 5× Denhardt's Solution, 5×SSC, 0.1% SDS and 100 μg/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra; DNA Cloning, supra; Nucleic Acid Hybridization, supra.

As used herein the term “adjuvant!” refers to any material that enhances the action of a drug, antigen, polynucleotide, vector or the like. It is intended, although not always explicitly stated, that molecules having similar biological activity as wild-type or purified peptide adjuvants (e.g., recombinantly produced or muteins thereof) and nucleic acid encoding these molecules are intended to be used within the spirit and scope of the invention.

As used herein, the term “treat” or “treatment” includes any of following: the prevention of infection or reinfection; the reduction or elimination of symptoms; and the reduction or complete elimination of a pathogen. Treatment may be effected prophylactically (prior to infection) or therapeutically (following infection).

By “vertebrate subject” is meant any member of the subphylum cordata, particularly mammals, including, without limitation, humans and other primates. The term does not denote a particular age. Thus, both adult and newborn individuals are intended to be covered.

General Overview of the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Cell-mediated immune responses are mediated through T lymphocytes (T cells), which are able to bind specific proteins (e.g., antigens) when the protein associates with a major histocompatibility complex (“MHC”), typically through an antigen presenting cell (“APC,” such as a dendritic cell or macrophage). The APC digests the antigen into a smaller-sized fragment and presents it on its surface bound to the APC's MHC.

T-cell receptors (TCR) are double stranded proteins present on the surface of T-cell and constitute the structurally variable antigen-specific receptor which recognizes and binds to the peptide-MHC complex presented by an APC. There are two known types of TCR, TCR2 (composed of an “α” and a “β” chain) and TCR1 (composed of a “γ” and a “δ” chain). The α and γ chains are each divided into variable (“V”), joining (“J”) and constant (“C”) regions. The β and δ chains each have V, J and C regions, and also have a diversity (“D”) region disposed between V and J. Over 95% of TCRs are αβ chain dimers, while approximately 5% are γδ chain dimers.

TCRs are expressed in enormous diversity, each TCR being specific for one or a few MHC-peptide complexes. The selectivity of TCRs is attributable to the numerous combinations of V, D and J regions, amplified by random hypervariability at the junctions between the genes for the V, D and J regions. Because this diversity, a variety of numerical nomenclatures have evolved. Widely accepted is the system described by Chothia et al. (1988) EMBO J. 7(12):3745-3755, in which the amino acids (“AA's”) of the β strand's V region (of particular interest in the present invention) are counted, starting from the conserved cysteine designated as having AA position 92, numerically downward towards the N-terminus. The corresponding conserved cysteine in Vα is designated as having AA position 90 in this system. TCRs have three complementarity determining regions: CDR1 which is disposed towards the N-terminal of the V region; CDR2 which is disposed somewhat in the middle of the V region (generally encompassing the 12th though 25th or 26th amino acids towards the C-terminus starting from the conserved WY framework at about amino acid 35); and CDR3 disposed across the V(D)J junctions.

Accumulating evidence also suggests the TCRs specific for epitopes on certain proteins may contribute to autoimmune disease. For example, TCRs specific for myelin basic protein (BP) may contribute to the pathogenesis of multiple sclerosis. In animals, a limited set of TCR α-chain variable and β-chain variable genes are utilized by TCRs specific for BP (see, e.g., Acha-Orbea et al. (1998) Cell 54:263-273) and monoclonal antibodies directed to these regions or synthetic peptides with sequences common to those TCR variable regions can both protect and treat animals with clinical sign of experimental autoimmune encephalomyelitis (EAE).

Additionally, vaccines which induce a cell-mediated immune response represent important strategies in combating T-cell mediated responses, such as autoimmune disorders. Current approaches to treating autoimmune disease involve administration of therapeutic agents which affect the immune system as a whole, thus creating unwanted side effects. Current T-cell vaccine therapies have demonstrated limited utility in provided relief for those conditions. Several groups have attempted to develop T-cell receptor (TCR) vaccines either using (1) recombinantly produced whole T-cell receptors (see, e.g., WO 99/60119 and WO 99/60120); (2) peptide fragments of TCRs, particularly fragments including the second complementarity determining region (CDR2), of TCRs (see, e.g., U.S. Pat. Nos. 5,614,192 and 5,776,459, both to Vanderbark); or (3) DNA-cardiotoxin injection of construct encoding variable regions of TCRs (see, e.g., U.S. Pat. No. 5,939,400 to Steinman et al.). These methods have shown inconsistent immune responses.

The present invention provides polynucleotides encoding T-cell receptors or fragments thereof; core carriers coated with these polynucleotides; particulate compositions made up of these polynucleotides; and methods of treating T-cell mediated diseases using these polynucleotides in a pharmaceutical composition as, for example, a nucleic acid (e.g., DNA) vaccine. The TCRs encoded by the polynucleotides are preferably specific TCRs associated with one or more autoimmune diseases.

Thus, the invention includes an efficient method of treating specific autoimmune diseases such as multiple sclerosis, psoriasis, rheumatoid arthritis, lupus and the like by inducing a suppressive immune response (e.g., humoral and/or cellular) against T-cells (e.g., specific V-beta receptor types) that are prevalent or over-expressed at the site of disease lesion. These over-expressed TCRs are first identified and polynucleotides encoding whole or variable regions of these specific TCRs are then constructed. Further, unlike known TCR vaccines, the polynucleotides of the present invention can encode whole T-cell receptors, wherein the component chains of the T-cell receptor encoded by the polynucleotides are preferably refolded into their dimer configuration in vivo.

The polynucleotides of the present invention may be introduced into cells in vitro or in vivo, for example by transfection or by coating the polynucleotides onto particles and administering the coated particles to the cells. Alternatively, the polynucleotides may be provided in a particulate (e.g., powder) form, discussed more fully below and in the disclosure of International Publication Number WO 98/10750, which is incorporated by reference herein.

Advantages of the present invention include, but are not limited to, (i) in vivo production of immunogenic TCRs at or near the site of disease; (ii) eliminating the need to produce large quantities of correctly folded T-cell receptors outside the body; (ii) increasing immunogenicity by administering polynucleotides encoding larger proteins which are more immunogenic than smaller proteins; (iii) improving the consistency of a cell-mediated immune response; (iv) triggering the pathways that are normally involved in T-cell receptor mediated disease, e.g., autoimmune disorders and (v) treating T-cell mediated diseases by vaccination against over-expressed T-cell types.

T-Cell Receptor Mediated Diseases

The compositions and methods described herein are useful in treating a wide variety of T-cell receptor mediated diseases, for example autoimmune diseases. Autoimmune diseases are characterized by cytotoxic immune responses to epitopes on self antigens natively found in the diseased individual. The immune system of the individual then activates an inflammatory cascade aimed at cells and tissues presenting those specific self antigens. The destruction of the antigen, tissue, cell type, or organ attacked by the individual's own immune system gives rise to the symptoms of the disease.

Clinically significant autoimmune diseases include, for example, rheumatoid arthritis, multiple sclerosis, juvenile-onset diabetes, systemic lupus erythematosus, autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura. See e.g., Paul, W. E. (1993) Fundamental; Immunology, Third Edition, Raven Press, New York, Chapter 30, pp. 1033-1097; and Cohen et al. (1994) Autoimmune Disease Models, A Guidebook, Academic Press, 1994.

Autoimmune diseases are typically associated with an increased prevalence of specific TCRs. Thus, the condition to be treated will determine the choice of TCR sequences used in the compositions and methods described herein. Identification of TCRs associated with inflammatory disease allows therapies to be employed that inhibit the proinflammatory response of T-cells having such specific TCRs. It will be apparent from the teachings herein that identification of other specific TCRs (e.g., whole TCRs or variable regions such as the CDR2 region) that are preferentially expressed in any given autoimmune diseases can be conducted using methods known in the art, for example by sequence analysis or by single-strand conformation polymorphism (SSCP). (see, e.g., Yamamoto et al. (1992) Int. Immunol. 4:1219; Yamamoto et al (1996) Hum. Immunol. 48:23). Other methods of identifying TCRs include in vitro antigen-based assays to determine the variable region configuration of T cells that respond to a test antigen. These assays may utilize whole antigen or selected immunodominant peptides, using methods known in the art.

It will also be apparent from the teachings herein that, depending on the particular disease, various tissues may be employed for identifying the relevant TCRs. For neural disease such as MS, brain plaques or cerebrospinal fluid may be employed as a source of T cells. Similarly, for myasthenia gravis, muscle, thymus tissue or T-cells responsive to acetylcholine receptor may be employed. For rheumatoid arthritis, the synovial fluid may be employed.

Furthermore, many specific TCRs that are over-expressed in autoimmune diseases have previously been identified. For example, TCRs specific for human heat shock protein 60 and/or type II collagen (CII) appear to be over-represented in subjects afflicted with rheumatoid arthritis. Rheumatoid arthritis is an inflammatory, autoimmune disease affecting multiple systems, but primarily affecting multiple joints. The disease is accompanied by inflammation of the synovial membranes and other joint structures, muscle atrophy, bone erosion and rarefaction, and formation of pannus (fibrovascular inflammatory membrane) on the joint surfaces. Rheumatoid arthritis generally is quite painful and often severely debilitating.

Similarly, T cells reactive to myelin basic protein (MBP) and proteolipid protein (PLP) have been found in the cerebrospinal fluid and blood of patients with multiple sclerosis. MS is a chronic central nervous system disease and is the most common demyelinating disorder of the brain and spinal cord. See Martin et al., Immunological aspects of demyelinating diseases. Ann. Rev. Immunol. 10:153-187 (1992). T cell lines derived from patients with multiple sclerosis recognize an HLA-DR2-restricted epitope in residues 84-106 (Ota et al., Nature 346:183-187,(1989)). Additionally, Martin et al., J. Exp. Med. 173:19 (1991), identified an epitope in residues 87-106 that was recognized by multiple sclerosis T cells.

Certain T-cell receptors also appear to be over-expressed in subjects with diabetes mellitus. In vitro proliferation of T cells from diabetics appears to be stimulated by a 38-kD beta-cell granule protein. (Roep et al. (1990) Nature 345:632-634). Further, there is some evidence that T cell in diabetics may be recognizing insulin as an autoantigen. (McInerney et al. Diabetes Res Clin Pract. (2000) March;47(3):151-68).

Thus, one or more specific TCRs appear to be more prevalent in certain diseases. Identification of these TCRs allows for the design of anti-TCR nucleic acid (e.g., DNA) vaccines and methods of treating a wide variety of T-cell mediated diseases.

Polynucleotides

The polynucleotides of the present invention will comprise coding sequences for whole TCR molecules or fragments thereof, particularly fragments of the variable region. Preferably, the sequence will be sufficient to induce a suppressive, cross-reactive immune response against T cells displaying (encoding) the native TCRs upon administration. The polynucleotides may be formulated to include one or more TCR molecule or fragments thereof. Thus, it may be desirable in some cases to include multiple TCR coding sequences, wherein each recognizes the same epitope, a different epitope or a different epitope-MHC combination.

Nucleotide sequences selected for use in the present invention can be derived from known sources, for example, by isolating the same from cells containing a desired gene or nucleotide sequence using standard techniques. Similarly, the nucleotide sequences can be generated synthetically using standard modes of polynucleotide synthesis that are well known in the art. See, e.g., Edge et al. (1981) Nature 292:756-762; Nambair et al. (1994) Science 223:1299-1301; Jay et al. (1984) J. Biol. Chem. 259:6311-6317. Generally, synthetic oligonucleotides can be prepared by either the phosphotriester method as described by Edge et al., supra, and Duckworth et al. (1981) Nucleic Acids Res. 9:1691-1706, or the phosphoramidite method as described by Beaucage et al. (1981) Tet. Letts. 22:1859, and Matteucci et al. (1981) J. Am. Chem. Soc. 103:3185. Synthetic oligonucleotides can also be prepared using commercially available automated oligonucleotide synthesizers. The nucleotide sequences can thus be designed with appropriate codons for a particular amino acid sequence. In general, one will select preferred codons for expression in the intended host. The complete sequence is assembled from overlapping oligonucleotides prepared by standard methods and assembled into a complete coding sequence. See, e.g., Edge et al. (supra); Nambair et al. (supra) and Jay et al. (supra).

Another method for obtaining nucleic acid sequences for use herein is by recombinant means. Thus, a desired nucleotide sequence can be excised from a plasmid carrying the same using standard restriction enzymes and procedures. Site specific DNA cleavage is performed by treating with the suitable restriction enzyme (or enzymes) under conditions which are generally understood in the art, and the particulars of which are specified by manufacturers of commercially available restriction enzymes. If desired, size separation of the cleaved fragments may be performed by polyacrylamide gel or agarose gel electrophoresis using standard techniques.

Restriction cleaved fragments may be blunt ended by treating with the large fragment of E. coli DNA polymerase I (Klenow) in the presence of the four deoxynucleotide triphosphates (dNTPs) using standard techniques. The Klenow fragment fills in at 5′ single-stranded overhangs but digests protruding 3′ single strands, even though the four dNTPs are present. If desired, selective repair can be performed by supplying only one, or several, selected dNTPs within the limitations dictated by the nature of the overhang. After Klenow treatment, the mixture can be extracted with e.g. phenol/chloroform, and ethanol precipitated. Treatment under appropriate conditions with S1 nuclease or BAL-31 results in hydrolysis of any single-stranded portion.

Yet another convenient method for isolating specific nucleic acid molecules is by the polymerase chain reaction (PCR). Mullis et al. (1987) Methods Enzymol. 155:335-350. This technique uses DNA polymerase, usually a thermostable DNA polymerase, to replicate a desired region of DNA. The region of DNA to be replicated is identified by oligonucleotides of specified sequence complementary to opposite ends and opposite strands of the desired DNA to prime the replication reaction. The product of the first round of replication is itself a template for subsequent replication, thus repeated successive cycles of replication result in geometric amplification of the DNA fragment delimited by the primer pair used. This method also allows for the facile addition of nucleotide sequences onto the ends of the DNA product by incorporating these added sequences onto the oligonucleotide primers (see, e.g., PCR Protocols, A Guide to Methods and Applications, Innis et al (eds) Harcourt Brace Jovanovich Publishers, NY (1994)). PCR conditions used for each amplification reaction are empirically determined. A number of parameters influence the success of a reaction. Among them are annealing temperature and time, extension time, Mg²⁺ and ATP concentration, pH, and the relative concentration of primers, templates, and deoxyribonucleotides. One example of suitable PCR conditions is found below in the Examples.

A number of specific TCR molecule or TCR fragment peptide sequences, and nucleic acid sequences encoding those molecules or peptides, are known in the art, wherein the specific TCR molecule is associated with a disorder or disease state such as an autoimmune disorder. In particular, a number of suitable such peptide and nucleic acid sequences are disclosed in U.S. Pat. No. 6,207,645 to Howell et al., incorporated herein by reference. These and other known or readily identified sequences can be used to base or derive coding sequences for the TCR molecules or TCR fragment peptides used in the practice of the present invention. In addition, in some cases it may be useful to alter the sequence of the encoded TCR molecule or TCR fragment peptide in order to enhance the immunogenicity of such expression products. In this regard, minor changes can be engineered into the coding sequence for the subject molecules such that a different, yet substantially homologous expression product (protein or peptide molecule) is produced. Accordingly, the present invention includes polynucleotides containing coding sequences for TCR molecules or TCR fragment peptides that are substantially homologous to a native TCR sequence, for example polynucleotides that encode molecules having at least about 80-85%, preferably at least about 90% and most preferably at least about 95-98% sequence identity to the target native TCR molecule over the length of the relevant molecule.

Thus, once coding sequences for the desired TCR molecule(s) and/or TCR peptide fragment(s) have been prepared or isolated, such sequences can be cloned into any suitable vector or replicon. Numerous cloning vectors are known to those of skill in the art, and the selection of an appropriate cloning vector is a matter of choice. Ligations to other sequences are performed using standard procedures, known in the art.

As described in detail below, selected nucleotide sequences can be placed under the control of regulatory sequences such as a promoter, so that the sequence encoding the desired protein is transcribed into RNA in the host tissue transformed by a vector containing this expression construct.

In addition to promoters, it may be desirable to add other regulatory sequences which allow for regulation of the expression of protein sequences encoded by the delivered nucleotide sequences. Suitable additional regulatory sequences are known to those of skill in the art, and examples include those which cause the expression of a coding sequence to be turned on or off in response to a chemical or physical stimulus, including the presence of a regulatory compound. Other types of regulatory elements may also be present in the vector, for example, enhancer sequences.

An expression vector is constructed so that the particular coding sequence is located in the vector with the appropriate regulatory sequences such that the positioning and orientation of the coding sequence with respect to the control sequences allows the coding sequence to be transcribed under the “control” of the control sequences (i.e., RNA polymerase, which binds to the DNA molecule at the control sequences, transcribes the coding sequence). Modification of the sequences encoding the particular protein of interest may be desirable to achieve this end. For example, in some cases it may be necessary to modify the sequence so that it is attached to the control sequences with the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

Generally, nucleic acid molecules used in the subject methods contain coding regions with suitable control sequences and, optionally, ancillary nucleotide sequences which encode cytokines or other immune enhancing polypeptides. The nucleic acid molecules are generally prepared in the form of vectors which include the necessary elements to direct transcription and translation in a recipient cell.

Administration of Polynucleotides

The polynucleotides and ancillary substances described herein may be administered by any suitable method. In a preferred embodiment, described below, the polynucleotides are administered by coating them onto core carrier particles and then administering the coated particles to the subject or cells. However, the TCR-encoding polynucleotides may also be delivered using a viral vector or using non-viral systems, e.g., naked nucleic acid delivery.

Viral Vectors

A number of viral based systems have been used for gene delivery. For example, retroviral systems are known and generally employ packaging lines which have an integrated defective provirus (the “helper”) that expresses all of the genes of the virus but cannot package its own genome due to a deletion of the packaging signal, known as the psi sequence. Thus, the cell line produces empty viral shells. Producer lines can be derived from the packaging lines which, in addition to the helper, contain a viral vector which includes sequences required in cis for replication and packaging of the virus, known as the long terminal repeats (LTRs). The gene of interest can be inserted in the vector and packaged in the viral shells synthesized by the retroviral helper. The recombinant virus can then be isolated and delivered to a subject. (See, e.g., U.S. Pat. No. 5,219,740.) Representative retroviral vectors include but are not limited to vectors such as the LHL, N2, LNSAL, LSHL and LHL2 vectors described in e.g., U.S. Pat. No. 5,219,740, incorporated herein by reference in its entirety, as well as derivatives of these vectors, such as the modified N2 vector described herein. Retroviral vectors can be constructed using techniques well known in the art. See, e.g., U.S. Pat. No. 5,219,740; Mann et al. (1983) Cell 33:153-159.

Adenovirus based systems have been developed for gene delivery and are suitable for delivering the TCR polynucleotides described herein. Human adenoviruses are double-stranded DNA viruses which enter cells by receptor-mediated endocytosis. These viruses are particularly well suited for gene transfer because they are easy to grow and manipulate and they exhibit a broad host range in vivo and in vitro. For example, adenoviruses can infect human cells of hematopoietic, lymphoid and myeloid origin. Furthermore, adenoviruses infect quiescent as well as replicating target cells. Unlike retroviruses which integrate into the host genome, adenoviruses persist extrachromosomally thus minimizing the risks associated with insertional mutagenesis. The virus is easily produced at high titers and is stable so that it can be purified and stored. Even in the replication-competent form, adenoviruses cause only low level morbidity and are not associated with human malignancies. Accordingly, adenovirus vectors have been developed which make use of these advantages. For a description of adenovirus vectors and their uses see, e.g., Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; Rich et al. (1993) Human Gene Therapy 4:461-476.

Adeno-associated viral vectors (AAV) can also be used to administer the polynucleotides described herein. AAV vectors can be derived from any AAV serotype, including without limitation, AAV-1, AAV-2, AAV-3, AAV-4, AAV-5, AAVX7, etc. AAV vectors can have one or more of the AAV wild-type genes deleted in whole or in part, preferably the rep and/or cap genes, but retain one or more functional flanking inverted terminal repeat (ITR) sequences. A functional ITR sequence is generally deemed necessary for the rescue, replication and packaging of the AAV virion. Thus, an AAV vector includes at least those sequences required in cis for replication and packaging (e.g., a functional ITR) of the virus. The ITR need not be the wild-type nucleotide sequence, and may be altered, e.g., by the insertion, deletion or substitution of nucleotides, so long as the sequence provides for functional rescue, replication and packaging.

AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is bounded (5′ and 3′) with functional AAV ITR sequences. Suitable AAV constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Adjuvants

In some embodiments, the present invention may effectively be used with any suitable adjuvant or combination of adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); adjuvants formed from bacterial cell wall components such as adjuvants including lipopolysaccharides (e.g., lipid A or monophosphoryl lipid A (MPL), Imoto et al. (1985) Tet. Lett. 26:1545-1548), trehalose dimycolate (IDM), and cell wall skeleton (CWS); heat shock protein or derivatives thereof; adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, Staphylococcus aureus EDIN, and ADP-ribosylating bacterial toxin mutants such as CRM₁₉₇, a non-toxic diphtheria toxin mutant (see, e.g., Bixiler et al. (1989) Adv. Exp. Med. Biol. 251:175; and Constantino et al. (1992) Vaccine); saponin adjuvants such as Quil A (U.S. Pat. No. 5,057,540), or particles generated from saponins such as ISCOMs (immunostimulating complexes); chemokines and cytokines, such as interleukins (e.g., IL-1, IL-2, IL4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1-α and MIP-2, etc; muramyl peptides such as N-acetyl-muramyl-L-threonyl-D-isoglutamine (thr-MDP), N-acetyl-normuramyl-^{L}-alanyl-^{D}-isoglutamine (nor-MDP), N-acetylmuramyl-^{L}-alanyl-^{D}-isoglutaminyl-^{L}-alanine-2-(1′-2′-dipalmitoyl-sn-glycero-3 huydroxyphosphoiyloxy)-ethylamine (MTP-PE) etc.; adjuvants derived from the CpG family of molecules, CpG dinucleotides and synthetic oligonucleotides which comprise CpG motifs (see, e.g., Krieg et al. Nature (1995) 374:546, Medzhitov et al. (1997) Curr. Opin. Immunol. 9:4-9, and Davis et al. J. Immunol. (1998) 160:870-876) such as TCC ATG ACG TTC CTG ATG CT (SEQ ID NO:1) and ATC GAC TCT CGA GCG TTC TC (SEQ ID NO: 2); and synthetic adjuvants such as PCPP (Poly[di(carboxylatophenoxy)phosphazene) (Payne et al., Vaccines (1998) 16:92-98). Such adjuvants are commercially available from a number of distributors such as Accurate Chemicals; Ribi Immunochemicals, Hamilton, Mont.; GIBCO; Sigma, St. Louis, Mo.

The adjuvant may delivered individually or delivered in a combination of two or more adjuvants. In this regard, combined adjuvants may have an additive or a synergistic effect in promoting an immune response. A synergistic effect is one where the result achieved by combining two or more adjuvants is greater than one would expect than by merely adding the result achieved with each adjuvant when administered individually.

Conventional Pharmaceutical Preparations

Formulation of a preparation comprising the polynucleotides of the present invention, with or without addition of an adjuvant composition, can be carried out using standard pharmaceutical formulation chemistries and methodologies all of which are readily available to the ordinarily skilled artisan. For example, compositions containing one or more nucleic acid molecule (e.g., a plasmid containing a TCR coding sequence) can be combined with one or more pharmaceutically acceptable excipients or vehicles to provide a liquid preparation.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer, particularly for peptide, protein or other like molecules if they are to be included in the vaccine composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]N,N,N-trimethylammonium chloride), available under the tradename Lipofectin™ and DOTAP (1,2-bis(oleyloxy)-3-(trimethylammonio)propane), see, e.g., Felgner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolanine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publication No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated vaccine compositions will thus typically include a polynucleotide containing a sequence encoding a TCR molecule of interest in an amount sufficient to mount an immunological response. An appropriate effective amount can be readily determined by one of skill in the art. Such an amount will fall in a relatively broad range that can be determined through routine trials. For example, immune responses have been obtained using as little as 1 μg of DNA, while in other administrations, up to 2 mg of DNA has been used. It is generally expected that an effective dose of polynucleotides containing the genomic fragments will fall within a range of about 10 μg to 1000 μg, however, doses above and below this range may also be found effective. The compositions may thus contain from about 0.1% to about 99.9% of the polynucleotide molecules.

Administration of Conventional Pharmaceutical Preparations

Administration of the above-described pharmaceutical preparations can be effected in one dose, continuously or intermittently throughout the course of treatment. Delivery will most typically be via conventional needle and syringe for the liquid compositions and for liquid suspensions containing particulate compositions. In addition, various liquid jet injectors are known in the art and may be employed to administer the present compositions. Methods of determining the most effective means and dosages of administration are well known to those of skill in the art and will vary with the delivery vehicle, the composition of the therapy, the target cells, and the subject being treated. Single and multiple administrations can be carried out with the dose level and pattern being selected by the attending physician. It should be understood that more than one TCR sequence can be carried by the delivered polynucleotide construct. Alternatively, separate constructs each expressing one or more TCR molecules can also be delivered to a subject as described herein.

Furthermore, it is also intended that the polynucleotides delivered by the methods of the present invention be combined with other suitable compositions and therapies. For instance, in order to augment an immune response in a subject, the compositions and methods described herein can further include ancillary substances (e.g., adjuvants), such as pharmacological agents, cytokines, or the like. Ancillary substances may be administered, for example, as proteins or other macromolecules at the same time, prior to, or subsequent to, administration of the nucleic acid vaccines described herein. The nucleic acid molecule compositions may also be administered directly to the subject or, alternatively, delivered ex vivo, to cells derived from the subject, using methods known to those skilled in the art.

Particles

In one embodiment, the polynucleotides (e.g., DNA vaccines) and/or adjuvants are delivered using core carrier particles. Particle-mediated methods for delivering such nucleic acid preparations are known in the art. Thus, once prepared and suitably purified, the above-described nucleic acid molecules can be coated onto core carrier particles (e.g., core carriers) using a variety of techniques known in the art. Core carriers are selected from materials which have a suitable density in the range of particle sizes typically used for intracellular delivery from an appropriate particle-mediated delivery device. The optimum carrier particle size will, of course, depend on the diameter of the target cells.

For the purposes of the invention, tungsten, gold, platinum and iridium carrier particles can be used. Tungsten and gold particles are preferred. Tungsten particles are readily available in average sizes of 0.5 to 2.0 μm in diameter. Although such particles have optimal density for use in particle acceleration delivery methods, and allow highly efficient coating with DNA, tungsten may potentially be toxic to certain cell types. Gold particles or microcrystalline gold (e.g., gold powder A1570, available from Engelhard Corp., East Newark, N.J.) will also find use with the present methods. Gold particles provide uniformity in size (available from Alpha Chemicals in particle sizes of 1-3 μm, or available from Degussa, South Plainfield, N.J. in a range of particle sizes including 0.95 μm) and reduced toxicity.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in particle delivery devices.

Peptide adjuvants (e.g., cytokines), can also be coated onto suitable core carriers, e.g., gold or tungsten. For example, peptides can be attached to the core carrier by simply mixing the two components in an empirically determined ratio, by ammonium sulfate precipitation or other solvent precipitation methods familiar to those skilled in the art, or by chemical coupling of the peptide to the carrier particle. The coupling of L-cysteine residues to gold has been previously described (Brown et al., Chemical Society Reviews 9:271-311 (1980)). Other methods include, for example, dissolving the peptide antigen in absolute ethanol, water, or an alcohol/water mixture, adding the solution to a quantity of carrier particles, and then drying the mixture under a stream of air or nitrogen gas while vortexing. Alternatively, the peptide antigens can be dried onto core carriers by centrifugation under vacuum. Once dried, the coated particles can be resuspended in a suitable solvent (e.g., ethyl acetate or acetone), and triturated (e.g., by sonication) to provide a substantially uniform suspension.

Administration of Coated Particles

Following their formation, core carrier particles coated with the polynucleotides of the present invention and optionally including an adjuvant, are delivered to a subject using a transdermal injection (e.g., a particle-mediated delivery) technique.

Various particle delivery devices suitable for transdermal injection delivery are known in the art, and are all suited for use in the practice of the invention. Current device designs employ an explosive, electric or gaseous discharge to propel coated carrier particles toward target cells. The coated core carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 100.0 μg, more typically 0.01 to 10.0 μg of nucleic acid molecule per dose, and in the case of peptide or protein molecules is 1 μg to 5 mg, more typically 1 to 50 μg of peptide, depends on the subject to be treated. The exact amount necessary will vary depending on the age and general condition of the individual being immunized and the particular nucleotide sequence or peptide selected, as well as other factors. An appropriate effective amount can be readily determined by one of skill in the art upon reading the instant specification.

Thus, an effective amount of the polynucleotides will be that amount sufficient to bring about a suitable immune response in an immunized subject, and will fall in a relatively broad range that can be determined through routine trials. Preferably, the coated particles are delivered to suitable recipient cells in order to bring about an immune response (e.g., T-cell activation) in the treated subject.

Particulate Compositions

Alternatively, the polynucleotide encoding the selected TCR of interest can be formulated as a solid, particulate pharmaceutical composition. More particularly, formulation of particles comprising a nucleic acid molecule carrying a TCR sequence of interest can be carried out using standard pharmaceutical formulation chemistries and methodologies all of which are readily available to the reasonably skilled artisan. For example, nucleic acid molecules can be combined with one or more pharmaceutically acceptable excipients or vehicles to provide a vaccine composition. Auxiliary substances, such as wetting or emulsifying agents, pH buffering substances, and the like, may be present in the excipient or vehicle. These excipients, vehicles and auxiliary substances are generally pharmaceutical agents that do not themselves induce an immune response in the individual receiving the composition, and which may be administered without undue toxicity.

Pharmaceutically acceptable excipients include, but are not limited to, liquids such as water, saline, polyethyleneglycol, hyaluronic acid, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the composition will contain a pharmaceutically acceptable carrier that serves as a stabilizer, particularly for peptide, protein or other optional agents. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, carriers, stabilizers and other auxiliary substances is available in REMINGTON'S PHARMACEUTICAL SCIENCES (Mack Pub. Co., N.J. 1991), incorporated herein by reference.

The formulated compositions will include an amount of the polynucleotide encoding the TCR molecule of interest which is sufficient to mount an immunological response, as defined above. An appropriate effective amount can be readily determined by one of skill in the art. Such an amount will fall in a relatively broad range, generally within the range of about 0.1 μg to 25 mg or more of the TCR-encoding polynucleotide, and specific suitable amounts can be determined through routine trials. The compositions may contain from about 0.1% to about 99.9% of the polynucleotide. If an adjuvant is included in the composition, or the methods are used to provide a particulate adjuvant composition, the adjuvant will be present in a suitable amount as described above. The compositions are then prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in commonly owned International Publication No. WO 97/48485, incorporated herein by reference.

Single unit dosages or multidose containers, in which the particles may be packaged prior to use, can comprise a hermetically sealed container enclosing a suitable amount of the particles comprising the TCR-encoding polynucleotides and/or the selected adjuvant (e.g., the TCR vaccine composition). The particulate compositions can be packaged as a sterile formulation, and the hermetically sealed container can thus be designed to preserve sterility of the formulation until use in the methods of the invention. If desired, the containers can be adapted for direct use in a particle delivery device. Such containers can take the form of capsules, foil pouches, sachets, cassettes, and the like. Appropriate particle delivery devices are described herein above.

The container in which the particles are packaged can further be labelled to identify the composition and provide relevant dosage information. In addition, the container can be labelled with a notice in the form prescribed by a governmental agency, for example the Food and Drug Administration, wherein the notice indicates approval by the agency under Federal law of the manufacture, use or sale of the antigen, adjuvant (or vaccine composition) contained therein for human administration.

The particulate compositions (comprising the TCR polynucleotide and/or a selected adjuvant) can then be administered using a transdermal injection technique. Preferably, the particulate compositions will be delivered via a powder injection method, e.g., delivered from a needleless syringe system such as those described in commonly owned International Publication Nos. WO 94/24263, WO 96/04947, WO 96/12513, and WO 96/20022, all of which are incorporated herein by reference.

Delivery of particles from such devices is typically practised with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly sphencal particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and. 1.5 g/cm³, and injection velocities generally range between about 100 and 3,000 m/sec, or greater. With appropriate gas pressure, particles having an average diameter of 10-70 μm can be accelerated through the nozzle at velocities approaching the supersonic speeds of a driving gas flow.

If desired, these particle delivery devices can be provided in a preloaded condition containing a suitable dosage of the particles comprising the antigen of interest and/or the selected adjuvant. The loaded device can be packaged in a hermetically sealed container, which may further be labelled as described above.

Thus, the above-described methods can be used to obtain a particulate medicament (a “powder”) comprised of particles having a size ranging from about 10 to about 250 μm, preferably about 10 to about 150 μm, and most preferably about 20 to about 60 μm; and a particle density ranging from about 0.1 to about 25 g/cm³, and a bulk density of about 0.5 to about 3.0 g/cm³, or greater. The term “powder” as used herein refers to a composition that consists of substantially solid particles that can be delivered to a vertebrate subject via a transdermal injection technique, for example using a needleless syringe device. The particles that make up the powder can be characterized on the basis of a number of parameters including, but not limited to, average particle size, average particle density, particle morphology (e.g. particle aerodynamic shape and particle surface characteristics) and particle penetration energy (P.E.). Particle size, density and morphology are selected such that the solid, particulate pharmaceutical composition is a powder that is suitable for transdermal injection. In addition, the particles making up the particulate medicament of the present invention should demonstrate a baseline penetration energy suitable for a powder being delivered via a transdermal injection technique.

The average particle size of the powder can vary widely and will generally range from about 0.1 to about 250 μm, for example from about 10 to about 100 μm and more typically from about 20 to about 70 μm. However, it is preferable that the powder contain particles of a substantially homogenous average particle size. In this regard, the average particle size of the powder can be measured as a mass mean aerodynamic diameter (MMAD) using conventional techniques such as microscopic techniques (where particles are sized directly and individually rather than grouped statistically), absorption of gases, permeability, light obscuration or time of flight. If desired, automatic particle-size counters can be used (e.g. Aerosizer, Coulter Counter, HIAC Counter, or Gelman Automatic Particle Counter) to ascertain the average particle size.

The solid, particulate pharmaceutical compositions (medicaments) of the present invention will preferably be a powder comprised of particles have a size appropriate for high-velocity transdermal injection to a subject, typically across the stratum corneum or a transmucosal membrane. Thus, the mean mass aerodynamic diameter (MMAD) of the particles will range from about 0.1 to about 250 μm, however in practice, the MMAD may be from about 10 to about 100 μm, and preferably from about 10 to about 70 μm or from about 20 to about 70 μm. Generally, the particles will have a very tight size distribution, wherein less than 10% by weight of the particles have a diameter that falls outside of a plus/minus range of 5 μm of the MMAD, that is, less than 10% of the particles will have a diameter at least 5 μm greater than the MMAD or at least 5 μm less than the MMAD Preferably, no more than 5% by weight of the particles have a diameter which is greater than the MMAD by 5 μm or more. Also preferably, no more than 5% by weight of the particles have a diameter which is smaller than the MMAD by 5 μm or more.

Actual particle density or “absolute density” can be readily ascertained using known quantification techniques such as helium pycnometry and the like. Alternatively, envelope (“tap” or “bulk”) density measurements can be used to assess the density of a powder according to the invention. The envelope density of a powder produced in accordance with the present invention is generally from 0.5 to 25 g/cm³, preferably from 0.8 to 1.5 g/cm³.

Envelope density information is particularly useful in characterizing the density of objects of irregular size and shape. Envelope density is the mass of an object divided by its volume, where the volume includes that of its pores and small cavities but excludes interstitial space. A number of methods of determining envelope density are known in the art, including wax immersion, mercury displacement, water absorption and apparent specific gravity techniques. A number of suitable devices are also available for determining envelope density, for example, the GeoPyc™ Model 1360, available from the Micromeritics Instrument Corp. The difference between the absolute density and envelope density of a sample pharmaceutical composition provides information about the sample's percentage total porosity and specific pore volume.

Particle morphology, particularly the aerodynamic shape of a particle, can be readily assessed using standard light microscopy. It is preferred that the particles which make up the instant powders have a substantially spherical or at least substantially elliptical aerodynamic shape. It is also preferred that the particles have an axis ratio of 3 or less to avoid the presence of rod- or needle-shaped particles. These same microscopic techniques can also be used to assess the particle surface characteristics, e.g. the amount and extent of surface voids or degree of porosity.

Thus, it is preferred that the solid, particulate pharmaceutical compositions of the invention are a powder comprised of particles that have an envelope density ranging from about 0.1 to 2.5 g/cm³, preferably from about 0.8 to about 1.5 g/cm³. While the shape of the individual particles may vary when viewed under a microscope, the particles are preferably substantially spherical. The average aspect ratio (the ratio of the major axis:minor axis) is typically from 3:1 to 1:1, for example from 2:1 to 1:1. Most preferably, the aspect ratio is 1:1 and the particles are thus substantially spherical.

Particle penetration energies can be ascertained using a number of conventional techniques, for example a metallized film P.E. test. A metallized film material (e.g. a 125 μm polyester film having a 350 Å layer of aluminum deposited on a single side) is used as a substrate into which the powder is fired from a needleless syringe (e.g. the needleless syringe described in U.S. Pat. No. 5,630,796 to Bellhouse et a) at an initial velocity of about 100 to 3000 m/sec. The metallized film is placed, with the metal-coated side facing upwards, on a suitable surface.

A needleless syringe loaded with a powder is placed with its spacer contacting the film, and then fired. Residual powder is removed from the metallized film surface using a suitable solvent. Penetration energy is then assessed using a BioRad Model GS-700 imaging densitometer to scan the metallized film, and a personal computer with a SCSI interface and loaded with MultiAnalyst software (BioRad) and Matlab software (Release 5.1, The MathWorks, Inc.) is used to assess the densitometer reading. A program is used to process the densitometer scans made using either the transmittance or reflectance method of the densitometer. The penetration energy of the spray-coated powders should be equivalent to, or better than that of reprocessed mannitol particles of the same size (mannitol particles that are freeze-dried, compressed, ground and sieved according to the methods of commonly owned International Publication No. WO 97/48485, incorporated herein by reference).

Administration of Particulate Compositions

Following their formation, the particulate composition (e.g., powder) can be delivered transdermally to the tissue of a vertebrate subject using a suitable transdermal injection technique. Various particle delivery devices suitable for transdermal delivery of the substance of interest are known in the art, and will find use in the practice of the invention. A particularly preferred particle delivery technique employs a needleless syringe to fire solid drug-containing particles in controlled doses into and through intact skin and tissue. See, e.g., U.S. Pat. No. 5,630,796 to Bellhouse et al. which describes a needleless syringe (also known as “the PowderJect® needleless syringe device”). Other needleless syringe configurations are known in the art and are described herein.

Compositions containing a therapeutically effective amount of the powdered molecules described herein can be delivered to any suitable target tissue via the above-described particle delivery devices. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid molecules, delivery is preferably to, and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 μg/kg to 100 μg/kg of nucleic acid molecule per dose, depends on the subject to be treated. Doses for other pharmaceuticals, such as physiological active peptides and proteins, generally range from about 0.1 μg to about 20 mg, preferably 10 μg to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

Thus, a “therapeutically effective amount” of the present particulate compositions will be sufficient to bring about treatment or prevention of disease or condition symptoms, and will fall in a relatively broad range that can be determined through routine trials.

Below are examples of specific embodiments for carrying out the present invention. The examples are offered for illustrative purposes only, and are not intended to limit the scope of the present invention in any way.

### EXAMPLES

Efforts have been made to ensure accuracy with respect to numbers used (e.g., amounts, temperatures, etc.), but some experimental error and deviation should, of course, be allowed for.

### Example 1

### Nucleic Acid Immunization Using Polynucleotides Encoding Whole TCRs or TCR Fragments

In order to assess the specificity and effectiveness of nucleic acid immunization using TCR-encoding polynucleotides, the following studies are carried out.

TCRs that are over-expressed or more prevalent in autoimmune disorders are identified. Constructs encoding whole TCRs or variable region fragments (e.g., CDR-2 region) that specifically recognize myelin basic protein (3P) or type II collagen are generated. The constructs are coated onto 1-3 μm gold particles (Degussa Corp., South Plainfield, N.J.) using techniques described by Eisenbraun et al. (1993) DNA Cell Biol. 12:791-797. Briefly, DNA is affixed to gold particles by adding the 1-3 micron gold powder (Degussa) and an appropriate amount of polynucleotide (e.g., 2 μg per mg of gold powder) to a centrifuge tube containing spermidine. Polynucleotide and gold are coprecipitated by the addition CaCl₂ dropwise while vortexing, after which the precipitate is allowed to settle for several minutes. The gold/DNA precipitate is concentrated by centrifugation in a microcentrifuge, washed with absolute ethanol and resuspended in an appropriate amount of ethanol and polyvinyl pyrrilodine (PVP). The suspension is then transferred to a glass vial which is capped and immersed in a sonicating water bath to resolve clumps. The ethanol solution is then injected into Tefzel® tubing and the gold/DNA adheres to the side of the tubing using centrifugal force. The remaining ethanol is then ejected using a small, controlled flow of nitrogen gas. The tube is then dried with a flow of nitrogen gas, and then cut into cartridges. The cartridges are stored in a tightly capped glass scintillation vials with dessicant at 4° C. until use with a PowderJect® XR particle delivery device. The helium-pulse PowderJect® XR particle-mediated device has been previously described (see, U.S. Pat. No. 5,584,807) and is obtained from PowderJect Vaccines, Madison, Wis. In the vaccinations, each cartridge nominally contains 0.5 mg gold particles coated with 1 μg of plasmid DNA.

Animal exhibiting symptoms of the target autoimmune disease (e.g., multiple sclerosis for BP-specific TCRs and rheumatoid arthritis for CII-specific TCRs) are immunized by particle-mediated delivery using a single shot of gold beads coated with polynucleotide delivered from a PowderJect® XR particle delivery device. Animals are boosted 4 weeks after priming. Serum is collected at the time of boosting (4 week) and two weeks later (6 week).

### Example 2

### Characterization and Assessment of Particulate Medicaments Comprising Polynucleotides That Encode Whole TCR Molecules or TCR Fragment Peptides

Assessment of Effect on Artificial Film Targets

A functional test that measures many aspects of powder injection systems simultaneously has been designated as the “metallized film” or “penetration energy” (PE) test. It is based upon the quantitative assessment of the damage that particles can do to a precision thin metal layer supported by a plastic film substrate. Damage correlates to the kinetic energy and certain other characteristics of the particles. The higher the response from the test (i.e., the higher the film damage/disruption) the more energy the device has imparted to the particles. Either electrical resistance change measurement or imaging densitometry, in reflectance or transmission mode, provide a reliable method to assess device or formulation performance in a controllable and reproducible test.

The film test-bed has been shown to be sensitive to particle delivery variations of all major device parameters including pressure, dose, particle size distribution and material, etc. and to be insensitive to the gas. Aluminum of about 350 Angstrom thickness on a 125 μm polyester support is currently used to test devices operated at up to 60 bar.

Assessment of Impact Effect on Engineering Foam Targets

Another means of assessing particle performance when delivered via a transdermal injection technique (e.g., delivered from a needleless syringe device) is to gauge the effect of impact on a rigid polymethylimide foam (Rohacell 5 IIG, density 52 kg/m³, Rohm Tech Inc., Malden, Mass.). The experimental set-up for this test is similar to that used in the metallized film test. The depth of penetration is measured using precision calipers. For each experiment a processed mannitol standard is run as comparison and all other parameters such as device pressure, particle size range, etc., are held constant. Data also show this method to be sensitive to differences in particle size and pressure. Processed mannitol standard as an excipient for drugs has been proven to deliver systemic concentrations in preclinical experiments, so the relative performance measure in the foam penetration test has a practical in vivo foundation. Promising powders can be expected to show equivalent or better penetration to mannitol for anticipation of adequate performance in preclinical or clinical studies. This simple, rapid test has value as a relative method of evaluation of powders and is not intended to be considered in isolation.

Particle Attrition Test

A further indicator of particle performance is to test the ability of various candidate solid, particulate pharmaceutical compositions to withstand the forces associated with high-velocity transdermal injection techniques, that is, the forces from contacting particles at rest with a sudden, high velocity gas flow, the forces resulting from particle-to-particle impact as the powder travels through the needleless syringe, and the forces resulting from particle-to-device collisions also as the powder travels through the device. Accordingly, a simple particle attrition test has been devised which measures the change in particle size distribution between the initial composition, and the composition after having been delivered from a needleless syringe device.

The test is conducted by loading a particle composition into a needleless syringe as described above, and then discharging the device into a flask containing a carrier fluid in which the particular composition is not soluble (e.g. mineral oil, silicone oil, etc.). The carrier fluid is then collected, and particle size distribution in both the initial composition and the discharged composition is calculated using a suitable particle sizing apparatus, e.g., an AccuSizer® model 780 Optical Particle Sizer. Compositions that demonstrate less than about 50%, more preferably less than about 20% or about 10%, reduction in mean mass aerodynamic diameter (as determined by the AccuSizer apparatus) after device actuation are deemed suitable for use in the needleless syringe systems described herein.

Delivery to Human Skin in vitro and Transepidermal Water Loss

For a composition performance test that more closely parallels eventual practical use, candidate particle compositions can be injected into dermatomed, full thickness human skin samples. Replicate skin samples after injection can be placed on modified Franz diffusion cells containing 32° C. water, physiologic saline or buffer. Additives such as surfactants may be used to prevent binding to diffusion cell components and to maintain sink conditions. Two kinds of measurements can be made to assess performance of the formulation in the skin.

To measure physical effects, i.e. the effect of particle injection on the barrier function of skin, the transepidermal water loss (TEWL) can be measured. Measurement is performed at equilibrium (about 1 hour) using a Tewameter TM 210® (Courage & Khazaka, Koln, Ger) placed on the top of the diffusion cell cap that acts like a ˜12 mm chimney. Larger particles and higher injection pressures generate proportionally higher TEWL values in vitro and this has been shown to correlate with results in vivo. Upon particle injection in vitro TEWL values increased from about 7 to about 27 (g/m²h) depending on particle size and helium gas pressure. Helium injection without powder has no effect. In vivo, the skin barrier properties return rapidly to normal as indicated by the TEWL returning to pretreatment values in about 1 hour for most powder sizes. For the largest particles, 53-75 μm, skin samples show 50% recovery in an hour and full recovery by 24 hours.

These and other qualatative and quantitative tests can be used to assess the physical and functional suitability of the present particulate medicaments for use in transdermal injection. It is preferred, though not required, that the particles have the following characteristics: a substantially spherical shape (e.g. an aspect ratio as close as possible to 1:1 such as from 1.5:1); a smooth surface; less than 20% reduction in particle size using the particle attrition test; an envelope density as close as possible to the true density of the constituents (e.g. greater than about 0.8 g/ml); and a MMAD of about 20 to 70 μm with a narrow particle size distribution. The compositions may be a free-flowing powder (e.g. free flowing after 8 hours storage at 50% relative humidity and after 24 hours storage at 40% relative humidity). All of these criteria can be assessed using the above-described methods, and are further detailed in the following publications, incorporated herein by reference. Etzler et al (1995) Part. Part. Syst. Charact.12:217; Ghadiri, et al (1992) IFPRI Final Report, FRR 16-03 University of Surrey, UK; Bellhouse et al (1997) “Needleless delivery of drugs in dry powder form, using shock waves and supersonic gas flow,” Plenary Lecture 6, 21^{st} International Symposium on Shock Waves, Australia; and Kwon et al (1998) Pharm. Sci. suppl.1 (1), 103.

Accordingly, novel compositions for eliciting an immune response have been described. Methods of using these compositions have also been described. Although preferred embodiments of the subject invention have been described in some detail, it is understood that obvious variations can be made without departing from the spirit and the scope of the invention as defined by the appended claims.

What is claimed is:

1. A core carrier coated with a polynucleotide, said polynucleotide comprising a coding sequence for at least one T-cell receptor (TCR) operably linked to control elements such that the coding sequence can be transcribed and translated in vivo in a recipient cell to provide a refolded TCR molecule.

2. The core carrier of claim 1, wherein the TCR molecule comprises an α chain and a β chain.

3. The core carrier of claim 1, wherein the TCR molecule comprises a γ chain and a γ chain.

4. The core carrier of any one of claims 1-3, wherein T cells expressing the TCR molecule are preferentially associated with an autoimmune disorder.

5. The core carrier of claim 4, wherein the autoimmune disorder is selected from the group consisting of multiple sclerosis, psoriasis, rheumatoid arthritis and lupus.

6. The core carrier of any one of claims 1-5, wherein the core carrier is comprised of gold or tungsten.

7. A pharmaceutical composition, comprising the core carrier of any one of claims 1-6 and a pharmaceutically acceptable excipient.

8. A core carrier coated with a polynucleotide comprising a coding sequence for a CDR2 hypervariable region of a T-cell receptor (TCR) operably linked to control elements such that the coding sequence can be transcribed and translated in vivo in a recipient cell to provide a CDR2 peptide, wherein the CDR2 is from a T cell preferentially associated with an autoimmune disorder.

9. The core carrier of claim 8, wherein the carrier is comprised of gold or tungsten.

10. A pharmaceutical composition comprising the coated core carrier of any one of claims 8 or 9, and A pharmaceutically acceptable excipient.

11. The core carrier of claim 8, further comprising a second polynucleotide comprising a coding sequence for a TCR hypervariable region selected from the group consisting of CDR1 and CDR3.

12. The core carrier of claim 11, wherein the carrier is comprised of gold or tungsten.

13. The core carrier of any one of claims 8-12, wherein the autoimmune disorder is selected from the group consisting of multiple sclerosis, psoriasis, rheumatoid arthritis and lupus.

14. A solid, particulate pharmaceutical composition, said composition containing a polynucleotide comprising a coding sequence for at least one T-cell receptor (TCR) operably linked to control elements such that the coding sequence can be transcribed and translated in vivo in a recipient cell to provide a refolded TCR molecule.

15. The composition of claim 14, wherein the TCR molecule comprises an α chain and a β chain.

16. The composition of claim 14, wherein the TCR molecule comprises a γ chain and a δ chain.

17. The composition of any one of claims 14-16, wherein T cells expressing the TCR molecule are preferentially associated with an autoimmune disorder.

18. The composition of claim 17, wherein the autoimmune disorder is selected from the group consisting of multiple sclerosis, psoriasis, rheumatoid arthritis and lupus.

19. The composition of any one of claims 14-18, wherein said composition is comprised of a homogenous population of particles having an average particle diameter of about 0.1 to 250 μm and a density in the range of about 0.1 to 25 g/cm³.

20. A solid, particulate pharmaceutical composition, said composition containing a polynucleotide comprising a coding sequence for a CDR2 hypervariable region of a T-cell receptor (TCR) operably linked to control elements such that the coding sequence can be transcribed and translated in vivo in a recipient cell to provide a CDR2 peptide, wherein the CDR2 is from a T cell preferentially associated with an autoimmune disorder.

21. The composition of claim 20, further comprising a second polynucleotide comprising a coding sequence for a TCR hypervariable region selected from the group consisting of CDR1 and CDR3.

22. The composition of any one of claims 20 or 21, wherein the autoimmune disorder is selected from the group consisting of multiple sclerosis, psoriasis, rheumatoid arthritis and lupus.

23. The composition of any one of claims 20-22, wherein said composition is comprised of a homogenous population of particles having an average particle diameter of about 0.1 to 250 μm and a density in the range of about 0.1 to 25 g/cm³.

24. Use of a vector comprising a polynucleotide, said polynucleotide comprising a coding sequence for at least one T-cell receptor (TCR) operably linked to control elements such that the coding sequence can be transcribed and translated in vivo in recipient cells of a subject to provide a refolded TCR molecule in the manufacture of a medicament for eliciting a cross-reactive immune response against a T cell expressing a native form of said TCR, wherein said medicament is a particulate medicament suitable for transdermal injection into the said subject.

25. Use according to claim 24, wherein carrier particles having a nominal size of from about 0.5 to 5 μm are coated with the said vector to provide said particulate medicament.

26. Use according to claim 25, wherein the carrier particles are comprised of gold or tungsten.

27. Use according to claim 24, wherein the medicament is a solid, particulate pharmaceutical composition.

28. Use according to claim 27, wherein said composition is comprised of a homogenous population of particles having an average particle diameter of about 0.1 to 250 μm and a density in the range of about 0.1 to 25 g/cm³.

29. Use of a vector comprising a polynucleotide, said polynucleotide comprising a coding sequence for a CDR2 hypervariable region of a T-cell receptor (TCR) operably linked to control elements such that the coding sequence can be transcribed and translated in vivo in recipient cells of a subject to provide a CDR2 peptide, in the manufacture of a medicament for eliciting a cross-reactive immune response against a T cell expressing a native form of said TCR, wherein the CDR2 is from a T cell preferentially associated with an autoimmune disorder, and further wherein said medicament is a particulate medicament suitable for transdermal injection into the said subject.

30. Use according to claim 29, wherein carrier particles having a nominal size of from about 0.5 to 5 μm are coated with the said vector to provide said particulate medicament.

31. Use according to claim 30, wherein the carrier particles are comprised of gold or tungsten.

32. Use according to claim 29, wherein the medicament is a solid, particulate pharmaceutical composition.

33. Use according to claim 32, wherein said composition is comprised of a homogenous population of particles having an average particle diameter of about 0.1 to 250 μm and a density in the range of about 0.1 to 25 g/cm³.

34. A method for treating a T-cell mediated disease, the method comprising administering to a subject in need thereof an effective amount of the core carrier of any one of claims 1-13, whereby the polynucleotide is expressed in cells of said subject to provide a T-cell receptor (TCR) molecule or a CDR2 peptide in an amount sufficient to induce a cross-reactive immune response against a native TCR expressed by T cells mediating the T-cell mediated disease.

35. A method for treating a T-cell mediated disease, the method comprising administering to a subject in need thereof an effective amount of the solid, particulate pharmaceutical composition of any one of claims 14-23, whereby the polynucleotide is expressed in cells of said subject to provide a T-cell receptor (TCR) molecule or a CDR2 peptide in an amount sufficient to induce a cross-reactive immune response against a native TCR expressed by T cells mediating the T-cell mediated disease.
